(19) United States
(12) Patent Application Publication
(10) Pub. No.: US 2006/0100350 A1
(43) Pub. Date: May 11, 2006

(54) Rapid drying lacquers containing graft copolymers with segmented arms

(76) Inventors: Robert John Barsotti, Franklinville, NJ (US); Sheau-Hwa Ma, West Chester, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/983,462

(22) Filed: Nov. 8, 2004

(51) Int. Cl. A61K 9/16

(52) U.S. Cl. 524/543

(57) ABSTRACT

This invention relates to rapid drying lacquers that are particularly useful for automotive OEM refinish applications. The lacquer includes a novel acrylic graft copolymer with segmented (or block) arms. This invention is also directed to a process for producing coatings from the rapid drying lacquers. These lacquers are especially useful in providing for chip and humidity resistant coatings having improved adhesion.

### FIELD OF THE INVENTION

This invention relates to coating compositions and in particular to rapid drying lacquer coating compositions that are particularly useful for automotive refinishing.

### BACKGROUND OF THE INVENTION

To refinish or repair a finish on vehicle, such as a basecoat/clearcoat finish on automobile or truck bodies, different fast-drying coating compositions have been developed. A number of pigmented and clear air-dry acrylic lacquers have been used in the past to repair these basecoat/clearcoat finishes, but none meet the rapid drying times that are desired in combination with outstanding physical properties, such as chip and humidity resistance, and adhesion.

A key concern to a refinish customer which is typically the vehicle owner is that the coating in use has excellent physical properties such as chip and humidity resistance, and adhesion, as well as excellent aesthetic appearance.

Another key concern of the automobile and truck refinish industry is productivity, i.e., the ability to complete an entire refinish operation in the least amount of time. To accomplish a high level of productivity, any coatings applied need to have the ability to dry at ambient or elevated temperature conditions in a relatively short period of time. The term “dry” means that the resulting finish is physically dry to the touch in a relatively short period of time to minimize dirt pick-up, and, in the case of the basecoat, to allow for the application of the subsequent clear coat.

Current commercially available lacquers do not have these unique characteristics of rapidly drying under ambient temperature conditions along with the ability to form a finish having improved chip and humidity resistance and adhesion. It would be advantageous to have a lacquer with this unique combination of properties.

### SUMMARY OF THE INVENTION

This invention is directed to a coating composition, especially to a lacquer coating composition, comprising a film-forming binder and a volatile organic liquid carrier, wherein the binder contains a graft copolymer with segmented arm(s). More particularly, the graft copolymer has a polymeric backbone and segmented arm(s) comprising at least two polymeric segments, grafted at a single point thereof to the backbone, wherein

(a) the backbone is of polymerized ethylenically unsaturated monomer(s); and

(b) the segmented arm(s) are of polymerized ethylenically unsaturated monomer(s) that are attached to the backbone via a single point,

wherein the segments on each arm have a substantially different composition from their adjacent segment(s) and differ by functional groups (i.e., by presence of, type of, and/or relative concentration of functional groups), wherein the functional groups are selected from at least one of the group consisting of acid, hydroxyl or amine groups or mixtures of hydroxyl and additionally acid or amine groups.

The backbone may be the same or similar to one of the segments on the arms, with the proviso that any strong interacting/H-bonding functional groups such as acid groups are not present in both, at least not in the same concentration, or the backbone may be substantially different in the manner described above. Generally it is desired that the backbone be substantially different in composition from the arm segments, particularly from the outer arm segment, so that polymer-polymer interaction is weak to keep the viscosity low enough for practical application in a 6.0 pounds per gallon or less VOC system.

The lacquer composition is most suited for use as a pigmented basecoat lacquer in automotive refinish applications, on top of which a transparent (clear) topcoat is applied.

While this composition is preferably used as a lacquer coating which dries via solvent evaporation absent any substantial crosslinking occurring, it optionally may contain a polyisocyanate crosslinking agent for further improved film properties.

This invention is further directed to a process for producing a coating on the surface of a substrate, such as a vehicle body or part thereof, wherein the process comprises:

applying a layer of a lacquer coating composition on the substrate surface, which may be previously primed or sealed or otherwise treated, the lacquer comprising the aforesaid composition; and

drying the layer, preferably at ambient conditions, to form a coating on the surface of the substrate, on top of which a clearcoat can be applied.

Also included within the scope of this invention is a substrate coated with the lacquer coating composition disclosed herein.

### DETAILED DESCRIPTION OF THE INVENTION

As used herein:

“Lacquer” means a coating composition that dries primarily by solvent evaporation and does not require crosslinking to form a film having the desired physical properties.

All “molecular weights” are determined by gel permeation chromatography (GPC) using polystyrene as the standard.

“Tg” (glass transition temperature) of the polymer can be measured by differential scanning calorimetry (DSC) or it can be calculated as described by Fox in Bull. Amer. Physics Soc., 1, 3, page 123 (1956).

“Acrylic polymer” means a polymer comprised of polymerized “(meth)acrylate(s)” which mean acrylates and methacrylates, optionally copolymerized with other ethylenically unsaturated monomers, such as acrylamides, methacrylamides, acrylonitriles, methacrylonitriles, and vinyl aromatics such as styrene.

The present invention is directed to a pigmented or clear air-dry lacquer containing one or more acrylic polymers suited for various coating processes, such as automotive OEM and automotive refinish. The novel lacquer is particularly well suited for use in automotive refinishing, particularly as a refinish basecoat used for repairing or refinishing colored basecoat/clearcoat finishes on auto and truck bodies, on top of which a clearcoat is applied.

Advantageously, the air-dry lacquer coating compositions that are formed have excellent physical properties, such as excellent chip and humidity resistance and adhesion, without sacrificing desired fast dry properties at ambient temperatures and overall appearance, such as DOI (distinctness of image) and HOB (head on brightness).

The lacquer coating composition of this invention preferably contains about 5 to 95% by weight, based on the weight of the coating composition, of a film-forming binder which comprises a graft acrylic polymer with segmented arms and correspondingly about 5 to 95% by weight, based on the weight of the coating composition, of a volatile organic liquid carrier and optionally contains pigments in a pigment to binder weight ratio of about 0.1/100 to 200/100.

### Graft Copolymer with Segmented Arms

The graft copolymer used to formulate the lacquer of this invention has a weight average molecular weight ranging from about 5,000-200,000 and preferably about 10,000-100,000, and more preferably in a range from about 15,000-80,000.

The segmented graft copolymer is preferably an acrylic polymer and can be described as having a polymeric backbone and one or more side chains or so-called segmented arms attached to the backbone. In the present invention, the composition of each of the arm segments is different from those of the adjacent segments(s) to provide the unique properties desired. Each arm comprises at least two polymeric segments, preferably just 2 segments, i.e., an inner segment attached directly to the backbone and an outer segment attached to the inner segment. By “inner” and “outer” segments, it is meant the arm segments closest to and farthest from the backbone, respectively.

Preferably, the graft copolymer contains about 10-90%, preferably 20-80%, and more preferably 30-70%, by weight of the backbone and about 90-10%, preferably 80-20%, and more preferably 70-30%, by weight of segmented arms.

The segments on each arm can differ by presence of, type of and/or relative concentrations of functional groups. The functional groups used herein are capable of reacting or interacting with other molecules. The functional groups are selected from at least one of the following groups 1 to 5:

1) Hydroxyl groups (e.g., primary or secondary hydroxyl)

2) Acid groups (e.g., carboxyl groups);

3) Amine groups (e.g., primary, secondary, or tertiary amine);

4) Mixtures of hydroxyl and acid groups; or

5) Mixtures of hydroxyl and amine groups.

In addition, the backbone may be the same or similar to one of the segments on the arms or may be different.

The size of each arm segment will vary depending on the final properties desired. However, each arm segment should be substantially linear and contain on average at least 3 units of monomers and have a number average molecular weight greater than 300. In preferred embodiments, the number of monomers within a single segment is about 10 or more. Also in preferred embodiments, the weight average molecular weight of the total arm is in a range from about 1,000-40,000, preferably from about 1,500-30,000.

The presence of, concentration of, and type of functional groups on each arm segment and backbone will also vary depending on the particular attribute desired; however, if functional groups are present, the concentration should be such that at least 1% to 100%, more preferably at least 2-40% by weight, of the monomers used to form that given arm segment or backbone have functional groups. However, when strong interacting groups such as carboxylic acid groups, which tend to raise the viscosity of the coating composition, are present, the upper limit of monomers containing carboxylic acid groups is preferably only up to 30% by weight of that given arm segment or backbone.

Also, in the case where carboxylic acid groups are present, the preferred embodiment is to place the carboxylic acid group in either the backbone or in one of the arm segments, more preferably all in the backbone. However, when carboxylic acid groups are present in both, the relative concentration in one of the segments or backbone should be low enough to prevent hydrogen bonding interaction and viscosity build up in the coating composition.

In the present invention, a particularly useful embodiment comprises concentrating the functional groups on the outer segment of the arms, with the remaining arm segments, preferably just one, containing essentially no functional groups. The preferred functional group in the arm is a hydroxyl group. In this embodiment, the backbone also preferably contains a functional group which is different from that used in the arm. The preferred functional groups in the backbone are a mixture of carboxyl and hydroxyl groups.

By having the functional groups in arm segments separated from the backbone by a non-functional segment, it is believed that better compatibility of this acrylic copolymer with other film-forming components, such as polyester resins, in the lacquer is obtained, while also achieving the other properties desired, such as excellent chip, adhesion, and humidity resistance, while also maintaining the desired fast dry properties associated with high Tg coatings.

Also, by having segmented arms allows for the combination of potentially diverse polymer properties (such as functional/nonfunctional blocks and hard/soft blocks) into a single polymer chain. Non-functional/functional and/or hard/soft copolymer pairs in a single polymer chain can result in materials which possess performance attributes not found in any of the constituent segments.

For example, the functional groups concentrated on one portion of the polymeric arms, such as hydroxyl groups on the outer arm, as indicated above, have also been shown to improve the bonding of the lacquer to other coating layers and/or to improve compatibility of the polymer with other binder components in crosslinkable coating compositions. Also, by combining the stiffness or rigidity characteristic of hard materials with the compliance of soft materials, graft copolymers of this invention have exhibited advantageous properties, such as toughening of the coating or improving drying properties and metallic flake orientation in air-dry basecoat lacquer coatings.

The graft copolymers of this invention can be prepared in a variety of ways. In one embodiment, the graft copolymer is prepared from a macromonomer having a “segmented” (also referred to herein as a “block”) structure, which forms the segmented side arms of the graft copolymer, with only one terminal ethylenically unsaturated group for attachment to the backbone. The macromonomer, having the segmented structure and only one terminal ethylenically unsaturated group (or vinyl terminal group), is typically prepared first. It is then copolymerized with ethylenically unsaturated monomers chosen for the backbone composition to form the graft structure. This graft copolymer can be described as having a backbone having one or more, typically a plurality, of segmented macromonomer side chains or arms attached thereto.

In the macromonomer approach, the macromonomer that forms the arms is formed essentially like an AB block copolymer in that it contains at least two substantially linear polymeric segments of differing composition that are incorporated in the arm in a non-random manner, to form the individual arm segments. Each segment is formed from different ethylenically unsaturated monomers or monomer mixtures.

To form the segmented arms using this approach, the outer segment (which is also referred to herein as the “A segment”) is prepared first as a macromonomer, which has only one terminal ethylenically unsaturated group. The second segment or “B segment” (which forms the inner segment in this example) is then built on the segment A to produce the entire segmented arm. This is also a macromonomer having only one terminal ethylenically unsaturated group which is eventually polymerized into the backbone of the graft copolymer. Of course, additional segments may be added to the B segment until the desired number of segments is formed before the macromonomer is finally attached to the backbone. However, in the present invention, an average of two arm segments, A and B, are generally preferred and the present invention will now be discussed generally in this context. One skilled in the art would understand that the invention also is useful with more than two arm segments.

As indicated above, each segment is formed from different ethylenically unsaturated monomers or monomer mixtures. The outer arm segment A which is prepared first is a macromonomer generally comprised of polymerized acrylic monomer(s) and optionally other ethylenically unsaturated monomers, such as styrene, of which at least one monomer contains one of the functional groups listed above. The preferred functional groups used in the outer arm are hydroxyl groups.

The second segment B, which forms the inner segment is then built onto outer segment A to form the segmented macromonomer with only one terminal ethylenically unsaturated group which is eventually polymerized into the backbone of the graft copolymer, is prepared from a different set of monomers. The second segment is preferably a non-functional segment preferably comprised of polymerized acrylic monomer(s) and optionally other ethylenically unsaturated monomers such as styrene but which are non-functional, i.e., essentially free of functional groups. By “essentially free”, it is meant that the inner segment should contain less than 1% by weight, preferably zero percent by weight, of functionalized acrylic or other functionalized ethylenically unsaturated monomers, based on the total weight of the graft copolymer.

In an alternate embodiment, it may be desired to additionally formulate the outer arm segments so that it is soft relative to the inner arm segment. A combination of high Tg (e.g., methyl methacrylate and styrene) and low Tg monomers (e.g., butyl acrylate and 2-ethylhexyl acryl) can be used to form an arm with such a hard and soft segment.

For example, the outer functional arm segment can be formulated to have a calculated glass transition temperature (Tg) of no greater than 25° C., and preferably −60° to 25° C., while the inner segment of the arms is hard relative to the outer arm segment and can have a calculated glass transition temperature (Tg) of at least 30° C., and preferably 40° to 165° C.

Besides the arm segments, the backbone (also referred to herein as the “C” segment) is also preferably comprised of acrylic monomer(s) and optionally other ethylenically unsaturated monomers such as styrene. In a particular embodiment, the backbone like the outer segment of the arm preferably also contains at least one of the above functional groups but has at least one functional group which is different from the functional group(s) on the arm(s). This group will vary depending on the nature of the other binder components present in the lacquer coating; however, carboxylic acid groups as are listed below are generally preferred. A good combination of functional groups for the backbone is hydroxyl and carboxylic acid groups.

Even when using the macromonomer approach, the segmented macromonomers used to form the segmented arms on the graft copolymer can be prepared by a number of ways, including sequential addition of different monomers or monomer mixtures to living polymerization reactions such as anionic polymerization, group transfer polymerization, nitroxide-mediated free radical polymerization, atom transfer radical polymerization (ATRP) or reversible addition-fragmentation chain transfer (RAFT) polymerization, and finally converting the living end to a terminal polymerizable double bond, or by sequentially building one segment at a time using catalytic chain transfer agents as described below.

The catalytic chain transfer agent approach is the preferred method for making the segmented macromonomers of this invention. The other living polymerization approaches mentioned above often involve special and costly raw materials including special initiating systems and high purity monomers. Some of them have to be carried out under extreme conditions such as low moisture or low temperature. In addition, some of the initiating systems bring undesirable color, odor, metal complexes, or potentially corrosive halides into the product. Extra steps would be required to remove them. In the preferred method, the catalyst is used at extremely low concentration and has minimum impact on the quality of the product, and the synthesis can be conveniently accomplished in a one-pot process.

In the catalytic chain transfer agent approach, the segmented macromonomers are most conveniently prepared by a multi-step free radical polymerization process to ensure that the resulting segmented macromonomer only has one terminal ethylenically unsaturated group which will polymerize with the backbone monomers to form the graft copolymer. Such a process is taught, for example in U.S. Pat. No. 6,291,620 to Moad et al., hereby incorporated by reference in its entirety.

In the first step of the process, the first or outer segment A of the macromonomer is formed using a free radical polymerization method wherein ethylenically unsaturated monomers or monomer mixtures chosen for this segment are polymerized in the presence of cobalt catalytic chain transfer agents or other transfer agents that are capable of terminating the free radical polymer chain and forming a terminal polymerizable double bond in the process. The polymerization is preferably carried out at elevated temperature in an organic solvent or solvent blend using a conventional free radical initiator and Co (II) or (III) chain transfer agent.

Once the first macromonomer segment having the desired molecular weight and conversion is formed, the cobalt chain transfer agent is deactivated by adding a small amount of oxidizing agent such as hydroperoxide. The unsaturated monomers or monomer mixtures chosen for the next segment are then polymerized in the presence of the first segment and more initiator. This step, which can be referred to as a macromonomer step-growth process, is likewise carried out at elevated temperature in an organic solvent or solvent blend using a conventional polymerization initiator. Polymerization is continued until a macromonomer is formed of the desired molecular weight and desired conversion of the second segment into a two-segmented arm.

This latter growth step can be repeated using different monomers or mixture of monomers until the desired number of segments on the arms is formed. The final segment that is formed by the above process will have attached thereto a single terminal ethylenically unsaturated group which will be used to attach the macromonomer to the polymer backbone.

Preferred cobalt chain transfer agents are described in U.S. Pat. No. 4,680,352 to Janowicz et al and U.S. Pat. No. 4,722,984 to Janowicz, hereby incorporated by reference in their entirety. Most preferred cobalt chain transfer agents are pentacyano cobaltate (II), diaquabis (borondiflurodimethylglyoximato) cobaltate (II), and diaquabis (borondifluorophenylglyoximato) cobaltate (II). Typically these chain transfer agents are used at concentrations of about 2-5000 ppm based on the total weight of the monomer depending upon the particular monomers being polymerized and the desired molecular weight. By using such concentrations, macromonomers having the desired molecular weight can be conveniently prepared.

To make distinct arm segments (or blocks), the growth of each segment needs to occur to high conversion. Conversions are determined by size exclusion chromatography (SEC) via integration of polymer to monomer peak. For UV detection, the polymer response factor must be determined for each polymer/monomer polymerization mixture. Typical conversions can be 50% to 100% for each segment or block. Intermediate conversion can lead to block (segmented) copolymers with a transitioning (or tapering) block where the monomer composition gradually changes to that of the following block as the addition of the monomer or monomer mixture of the next block continues. This may affect polymer properties such as phase separation, thermal behavior and mechanical modulus and can be intentionally exploited to drive properties for specific applications. This may be achieved by intentionally terminating the polymerization when a desired level of conversion (e.g., >80%) is reached by stopping the addition of the initiators or immediately starting the addition of the monomer or monomer mixture of the next block along with the initiator.

After the macromonomer is formed as described above, solvent is optionally stripped off and the backbone monomers are added to the macromonomer along with additional solvent and polymerization initiator, in order to prepare the graft copolymer structure by conventional free radical polymerization methods. The backbone monomers can be copolymerized with the macromonomers via the single terminal unsaturated group of the macromonomer using any of the conventional azo or peroxide type initiators and organic solvents as described below. The backbone so formed contains polymerized ethylenically unsaturated monomers and any of the monomers including those with functional groups listed below for use in the macromonomer can be used.

Polymerization is generally continued in the same pot at the reflux temperature of the reaction mixture until a graft copolymer is formed having the desired molecular weight.

Besides the macromonomer approach, an alternative method for preparing the graft copolymer of this invention involves reversing some of the steps. The backbone with a desired composition and molecular weight and having a proper concentration of some functional groups that are capable of initiating a living polymerization process or some precursor groups that may be converted to such initiating groups may be synthesized first. Off of these initiating groups, arms of desired segmented structure may be built in a sequential manner by a living polymerization process. As an example, a proper level of 4-(alpha-bromomethyl) styrene may be copolymerized into a backbone composition. Then an atom transfer radical polymerization (ATRP) process may be used to build the segments from the benzyl bromide groups to form the segmented arms of this invention. Another example of an alternative method include synthesizing a segmented copolymer (arms) using one of the living polymerization processes mentioned above and terminating the polymer chain with a reactive group such as carboxylic acid first. The segmented arms are then grafted onto a backbone polymer having a coreactive group such as epoxy. The segmented arms are attached to the backbone through an ester linkage.

Typical solvents that can be used to form the macromonomer or the graft copolymer are alcohols, such as methanol, ethanol, n-propanol, and isopropanol; ketones, such as acetone, butanone, pentanone, and hexanone; alkyl esters of acetic, propionic, and butyric acids, such as ethyl acetate, butyl acetate, and amyl acetate; ethers, such as tetrahydrofuran, diethyl ether, and ethylene glycol and polyethylene glycol monoalkyl and dialkyl ethers such as cellosolves and carbitols; and, glycols such as ethylene glycol and propylene glycol; and mixtures thereof.

Any of the commonly used azo or peroxide type polymerization initiators can be used for preparation of the macromonomer or graft copolymer provided it has solubility in the solution of the solvents and the monomer mixture, and has an appropriate half life at the temperature of polymerization. “Appropriate half life” as used herein is a half-life of about 10 minutes to 4 hours. Most preferred are azo type initiators such as 2,2′-azobis (isobutyronitrile), 2,2′-azobis (2,4-dimethylvaleronitrile), 2,2′-azobis (methylbutyronitrile), and 1,1′-azobis (cyanocyclohexane). Examples of peroxy based initiators are benzoyl peroxide, lauroyl peroxide, t-butyl peroxypivalate, t-butyl peroctoate which may also be used provided they do not adversely react with the chain transfer agents under the reaction conditions for macromonomers.

Generally, monomers that may be polymerized using the methods of this invention include at least one monomer selected from the group consisting of unsubstituted or substituted alkyl acrylates, such as those having 1-20 carbon atoms in the alkyl group, alkyl methacrylate, such as those having 1-20 carbon atoms in the alkyl group, cycloaliphatic acrylates, cycloaliphatic methacrylates, aryl acrylates, aryl methacrylates, other ethylenically unsaturated monomers such as acrylonitriles, methacrylonitriles, acrylamides, methacrylamides, N-alkylacrylamides, N-alkylmethacrylamides, N,N-dialkylacrylamides, N,N-dialkylmethacrylamides, vinyl aromatics such as styrene, and combinations thereof. Functionalized versions of these monomers and their relative concentrations are especially useful in differentiating the segments of the arms and the backbone, as will be discussed further hereinbelow.

Specific monomers or comonomers that have no special functional groups and may be used in this invention include various non-functional acrylic monomers such as methyl methacrylate, ethyl methacrylate, propyl methacrylate (all isomers), butyl methacrylate (all isomers), 2-ethylhexyl methacrylate; isobornyl methacrylate, methacrylonitrile, methyl acrylate, ethyl acrylate, propyl acrylate (all isomers), butyl acrylate (all isomers), 2-ethylhexyl acrylate, isobornyl acrylate, acrylonitrile, etc, and optionally other ethylenically unsaturated monomers, e.g., vinyl aromatics such as styrene, alpha-methyl styrene, t-butyl styrene, and vinyl toluene, etc.

As for the functional groups, examples of monomers that can be used to introduce primary or secondary hydroxyl groups into the graft copolymer of this invention and differentiate the segments of the arms and/or backbone from each other include hydroxyl functional acrylic monomers such hydroxyl alkyl (meth)acrylates having 1-4 atoms in the alkyl group including 2-hydroxyethyl methacrylate (primary), hydroxypropyl methacrylate (all isomers, primary and secondary), hydroxybutyl methacrylate (all isomers, primary and secondary), 2-hydroxyethyl acrylate (primary), hydroxypropyl acrylate (all isomers, primary and secondary), hydroxybutyl acrylate (all isomers, primary and secondary), other hydroxy alkyl acrylates and methacrylates, and the like.

To introduce acid groups into the graft copolymer at the appropriate backbone or arm segment, acid-functional monomers can be used. Carboxylic acid functional monomers are generally preferred for better compatibility with other binder components in the lacquer coating composition. The most commonly used carboxyl group containing monomers are methacrylic acid and acrylic acid. Others include vinyl benzoic acid (all isomers), alpha-methylvinyl benzoic acid (all isomers), and the diacids such as maleic acid, fumaric acid, itaconic acid, and their anhydride form that can be hydrolyzed to the carboxylic acid groups after the polymers are made. Of course, a low level of other types of acid groups, such as sulfonic acid or phosphoric acid may be used.

Typically useful amine functional monomers which can be used to introduce primary, secondary and/or tertiary amine groups are aminoalkyl (meth)acrylates, such as tertiarybutylaminoethyl (meth)acrylate, N-methylaminoethyl (meth)acrylate and diethylaminoethyl (meth)acrylate.

To form the high and low Tg segments, such monomers as methyl methacrylate, isobornyl acrylate, cyclohexyl methacrylate, and t-butyl styrene contribute to high Tg, whereas such softening monomers as butyl acrylate and 2-ethylhexyl acrylate contribute to low Tg.

As indicated above, the choice of monomers and monomer mixtures for each segment and the backbone, the segment size, overall ratios of monomers used to form the segmented arms and the backbone, and molecular weights, and nature of each segment and the backbone will vary so as to provide the particular attribute desired for a particular application.

Particularly useful graft copolymers include the following:

a graft acrylic polymer having a backbone of polymerized (meth)acrylate monomers, styrene monomers, (meth)acrylic acid monomers, and hydroxy-functional (meth)acrylate, and branches of polymerized macromonomers having a weight average molecular weight of about 1,000-40,000 and containing two segments, an outer segment of polymerized non-functional alkyl (meth)acrylate monomers and hydroxy alkyl (meth)acrylate monomers, and an inner segment of non-functional alkyl (meth)acrylates. One particularly useful polymer comprises a backbone of polymerized methyl methacrylate, hydroxy ethyl acrylate, acrylic acid, and butyl acrylate and the segmented macromonomer chain comprises polymerized ethyl hexyl methacrylate and hydroxy ethyl acrylate in the outer segment and polymerized butyl methacrylate and methyl methacrylate in the inner segment.

The novel coating composition of the present invention can contain, as part of the binder, in the range of about 2 to 100% by weight, preferably about 5 to 80% by weight, and even more preferably in the range of from 10 to 70% by weight of graft copolymer with segmented arms, all weight percentages being based on the total weight of the binder.

### Other Binder Materials

In addition to the graft copolymer with segmented arms, the coating composition can also include, as part of the binder, 0 to 98% by weight, preferably in the range of 20 to 95%, and even more preferably from 30 to 90% by weight of an acrylic polymer, polyester, alkyd resin, acrylic alkyd resin, cellulose acetate butyrate, an iminated acrylic polymer, ethylene vinyl acetate co-polymer, nitrocellulose, plasticizer or a combination thereof, all weight percentages being based on the total weight of the binder.

Useful acrylic polymers are conventionally polymerized from a monomer mixture that can include one or more of the following monomers: an alkyl acrylate; an alkyl methacrylate; a hydroxy alkyl acrylate, a hydroxy alkyl methacrylate; acrylic acid; methacrylic acid; styrene; alkyl amino alkyl acrylate; and alkyl amino alkyl methacrylate, and mixtures thereof; and one or more of the following drying oils: vinyl oxazoline drying oil esters of linseed oil fatty acids, tall oil fatty acids, and tung oil fatty acids.

Suitable iminated acrylic polymers can be obtained by reacting acrylic polymers having carboxyl groups with propylene imine.

Useful polyesters include the esterification product of an aliphatic or aromatic dicarboxylic acid, a polyol, a diol, an aromatic or aliphatic cyclic anhydride and a cyclic alcohol. One such polyester is the esterification product of adipic acid, trimethylol propane, hexanediol, hexahydrophathalic anhydride and cyclohexane dimethylol.

Other polyesters that are useful in the present invention are branched copolyester polyols. One particularly preferred branched polyester polyol is the esterification product of dimethylolpropionic acid, pentaerythritol and epsilon-caprolactone. These branched copolyester polyols and the preparation thereof are further described in WO 03/070843 published Aug. 28, 2003, which is hereby incorporated by reference.

Suitable cellulose acetate butyrates are supplied by Eastman Chemical Co., Kingsport, Tenn. under the trade names CAB-381-20 and CAB-531-1 and are preferably used in an amount of 0.1 to 20% by weight based on the weight of the binder.

A suitable ethylene-vinyl acetate co-polymer (wax) is supplied by Honeywell Specialty Chemicals—Wax and Additives, Morristown, N.J., under the trade name A-C 405 (T) Ethylene—Vinyl Acetate Copolymer.

Suitable nitrocellulose resins preferably have a viscosity of about ½-6 seconds. Preferably, a blend of nitrocellulose resins is used. Optionally, the lacquer can contain ester gum and castor oil.

Suitable alkyd resins are the esterification products of a drying oil fatty acid, such as linseed oil and tall oil fatty acid, dehydrated castor oil, a polyhydric alcohol, a dicarboxylic acid and an aromatic monocarboxylic acid. One preferred alkyd resin is a reaction product of an acrylic polymer and an alkyd resin.

Suitable plasticizers include butyl benzyl phthalate, dibutyl phthalate, triphenyl phosphate, 2-ethylhexylbenzyl phthalate, dicyclohexyl phthalate, diallyl toluene phthalate, dibenzyl phthalate, butylcyclohexyl phthalate, mixed benzoic acid and fatty oil acid esters of pentaerythritol, poly(propylene adipate) dibenzoate, diethylene glycol dibenzoate, tetrabutylthiodisuccinate, butyl phthalyl butyl glycolate, acetyltributyl citrate, dibenzyl sebacate, tricresyl phosphate, toluene ethyl sulfonamide, the di-2-ethyl hexyl ester of hexamethylene diphthalate, and di(methyl cyclohexyl) phthalate. One preferred plasticizer of this group is butyl benzyl phthalate.

If desired, the lacquer can include metallic driers, chelating agents, or a combination thereof. Suitable organometallic driers include cobalt naphthenate, copper naphthenate, lead tallate, calcium naphthenate, iron naphthenate, lithium naphthenate, lead naphthenate, nickel octoate, zirconium octoate, cobalt octaoate, iron octoate, zinc octoate, and alkyl tin dilaurates, such as dibutyl tin dilaurate. Suitable chelating agents include aluminum monoisopropoxide monoversatate, aluminum (monoisopropyl)phthalate, aluminum diethoxyethoxide monoversatate, aluminum trisecondary butoxide, aluminum diisopropoxide monoacetacetic ester chelate and aluminum isopropoxide.

If the lacquer is to be used as a clearcoat for the exterior of automobiles and trucks, about 0.1 to 5% by weight, based on the weight of the total weight of the binder, of an ultraviolet light stabilizer or a combination of ultraviolet light stabilizers and absorbers can be added to improve the weatherability of the composition. These stabilizers include ultraviolet light absorbers, screeners, quenchers and specific hindered amine light stabilizers. Also, about 0.1 to 5% by weight, based on the total weight of the binder, of an antioxidant can be added. Most of the foregoing stabilizers are supplied by Ciba Specialty Chemicals, Tarrytown, N.Y.

Additional details of the foregoing additives are provided in U.S. Pat. Nos. 3,585,160, 4,242,243, 4,692,481, and US Re 31,309, which are hereby incorporated by reference.

### Pigments

If desired, the novel composition can be pigmented to form a colored mono coat, basecoat, primer or primer surfacer. Generally, pigments are used in a pigment to binder weight ratio (P/B) of 0.1/100 to 200/100; preferably, for base coats in a P/B of 1/100 to 50/100. If used as primer or primer surfacer higher levels of pigment are used, e.g., 50/100 to 200/100. The pigments can be added using conventional techniques, such as sand-grinding, ball milling, attritor grinding, two roll milling to disperse the pigments. The mill base is blended with the film-forming constituents.

Any of the conventional pigments used in coating compositions can be utilized in the composition such as the following: metallic oxides, metal hydroxide, metal flakes, chromates, such as lead chromate, sulfides, sulfates, carbonates, carbon black, silica, talc, china clay, phthalocyanine blues and greens, organo reds, organo maroons, pearlescent pigments and other organic pigments and dyes. If desired, chromate-free pigments, such as barium metaborate, zinc phosphate, aluminum triphosphate and mixtures thereof, can also be used.

Suitable flake pigments include bright aluminum flake, extremely fine aluminum flake, medium particle size aluminum flake, and bright medium coarse aluminum flake; mica flake coated with titanium dioxide pigment also known as pearl pigments. Suitable colored pigments include titanium dioxide, zinc oxide, iron oxide, carbon black, mono azo red toner, red iron oxide, quinacridone maroon, transparent red oxide, dioxazine carbazole violet, iron blue, indanthrone blue, chrome titanate, titanium yellow, mono azo permanent orange, ferrite yellow, mono azo benzimidazolone yellow, transparent yellow oxide, isoindoline yellow, tetrachloroisoindoline yellow, anthanthrone orange, lead chromate yellow, phthalocyanine green, quinacridone red, perylene maroon, quinacridone violet, pre-darkened chrome yellow, thio-indigo red, transparent red oxide chip, molybdate orange, and molybdate orange red.

### Liquid Carrier

The lacquer of the present invention can further, and typically does, contain at least one volatile organic solvent as the liquid carrier to disperse and/or dilute the above ingredients and form a coating composition having the desired properties. The solvent or solvent blends are typically selected from the group consisting of aromatic hydrocarbons, such as, petroleum naphtha or xylenes; ketones, such as, methyl amyl ketone, methyl isobutyl ketone, methyl ethyl ketone or acetone; esters, such as butyl acetate or hexyl acetate; glycol ether esters, such as, propylene glycol monomethyl ether acetate; and alcohols, such as isopropanol and butanol. The amount of organic solvent added depends upon the desired solids level, desired rheological (e.g., spray) properties, as well as the desired amount of VOC of the lacquer.

The total solids level of the coating of the present invention can vary in the range of from 5 to 95%, preferably in the range of from 7 to 80% and more preferably in the range of from 10 to 60%, all percentages being based on the total weight of the coating composition.

### Optional Crosslinking Component

If the novel composition is used as a clear coating composition, a crosslinking component is generally known to provide the improved level of durability and weatherability required for automotive and truck topcoats. Typically, polyisocyanates are used as the crosslinking agents. Suitable polyisocyanate has on average 2 to 10, alternately 2.5 to 8 and further alternately 3 to 8 isocyanate functionalities. Typically the coating composition has, in the binder, a ratio of isocyanate groups on the polyisocyanate in the crosslinking component to crosslinkable groups (e.g., hydroxyl and/or amine groups) of the branched acrylic polymer ranges from 0.25/1 to 3/1, alternately from 0.8/1 to 2/1, further alternately from 1/1 to 1.8/1.

Examples of suitable polyisocyanates include any of the conventionally used aromatic, aliphatic or cycloaliphatic di-, tri- or tetra-isocyanates, including polyisocyanates having isocyanurate structural units, such as, the isocyanurate of hexamethylene diisocyanate and isocyanurate of isophorone diisocyanate; the adduct of 2 molecules of a diisocyanate, such as, hexamethylene diisocyanate; uretidiones of hexamethylene diisocyanate; uretidiones of isophorone diisocyanate or isophorone diisocyanate; isocyanurate of meta-tetramethylxylylene diisocyanate; and a diol such as, ethylene glycol.

Polyisocyanates functional adducts having isocyanaurate structural units can also be used, for example, the adduct of 2 molecules of a diisocyanate, such as, hexamethylene diisocyanate or isophorone diisocyanate, and a diol such as ethylene glycol; the adduct of 3 molecules of hexamethylene diisocyanate and 1 molecule of water (available under the trademark Desmodur® N from Bayer Corporation of Pittsburgh, Pa.); the adduct of 1 molecule of trimethylol propane and 3 molecules of toluene diisocyanate (available under the trademark Desmodur® L from Bayer Corporation); the adduct of 1 molecule of trimethylol propane and 3 molecules of isophorone diisocyanate or compounds, such as 1,3,5-triisocyanato benzene and 2,4,6-triisocyanatotoluene; and the adduct of 1 molecule of pentaerythritol and 4 molecules of toluene diisocyanate.

The coating composition containing a crosslinking component preferably includes one or more catalysts to enhance crosslinking of the components on curing. Generally, the coating composition includes in the range of from 0.01 to 5% by weight, based on the total weight of the binder.

Suitable catalysts for polyisocyanate can include one or more tin compounds, tertiary amines or a combination thereof. Suitable tin compounds include dibutyl tin dilaurate, dibutyl tin diacetate, stannous octoate, and dibutyl tin oxide. Dibutyl tin dilaurate is preferred. Suitable tertiary amines include triethylene diamine. One commercially available catalyst that can be used is Fastcat® 4202 dibutyl tin dilaurate sold by Elf-Atochem North America, Inc. Philadelphia, Pa. Carboxylic acids, such as acetic acid, may be used in conjunction with the above catalysts to improve the viscosity stability of two component coatings.

### Application

In use, a layer of the novel composition is typically applied to a substrate by conventional techniques, such as, spraying, electrostatic spraying, roller coating, dipping or brushing. Spraying and electrostatic spraying are preferred application methods. When used as a pigmented coating composition, e.g., as a basecoat or a pigmented top coat, the coating thickness can range from 10 to 85 micrometers, preferably from 12 to 50 micrometers and when used as a primer, the coating thickness can range from 10 to 200 micrometers, preferably from 12 to 100 micrometers. When used as a clear coating, the thickness is in the range of from 25 micrometers to 100 micrometers. The coating composition can be dried at ambient temperatures or can be dried upon application for about 2 to 60 minutes at elevated drying temperatures ranging from about 50° C. to 100° C.

In a typical clearcoat/basecoat application, a layer of conventional clear coating composition is applied over the basecoat of the novel composition of this invention by the above conventional techniques, such as, spraying or electrostatic spraying. Generally, a layer of the basecoat is flashed for 1 minute to two hours under ambient or elevated temperatures before the application of the clear coating composition or dried at elevated temperatures shown above. Suitable clear coating compositions can include two-pack isocyanate crosslinked compositions, such as 72200S ChromaPremier® Productive Clear blended with an activator, such as 12305S ChromaPremier®Activator, or 3480S Low VOC Clear composition activated with 194S Imron Select® Activator. Isocyanate free crosslinked clear coating compositions, such as 1780S Iso-Free Clearcoat activated with 1782S Converter and blended with 1775S Mid-Temp Reducer are also suitable. Suitable clear lacquers can include 480S Low VOC Ready to Spray Clear composition. All the foregoing clear coating compositions are supplied by DuPont (E.I. Dupont de Nemours and Company, Wilmington, Del.).

If the coating composition of the present invention contains a crosslinking agent, such as a polyisocyanate, the coating composition can be supplied in the form of a two-pack coating composition in which the first-pack includes the branched acrylic polymer and the second pack includes the crosslinking component, e.g., a polyisocyanate. Generally, the first and the second packs are stored in separate containers and mixed before use. The containers are preferably sealed air tight to prevent degradation during storage. The mixing may be done, for example, in a mixing nozzle or in a container. When the crosslinking component contains, e.g., a polyisocyanate, the curing step can take place under ambient conditions, or if desired, it can take place at elevated baking temperatures.

For a two-pack coating composition, the two packs are mixed just prior to use or 5 to 30 minutes before use to form a potmix. A layer of the potmix is typically applied to a substrate by the above conventional techniques. If used as a clear coating, a layer is applied over a metal substrate, such as, automotive body, which is often pre-coated with other coating layers, such as, an electrocoat primer, primer surfacer and a basecoat. The two-pack coating composition may be dried and cured at ambient temperatures or may be baked upon application for 10 to 60 minutes at baking temperatures ranging from 80° C. to 160° C. The mixture can also contain pigments and can be applied as a mono coat or a basecoat layer over a primed substrate or as a primer layer.

The coating composition of the present invention is suitable for providing coatings on variety of substrates. Typical substrates, which may or may not be previously primed or sealed, for applying the coating composition of the present invention include automobile bodies, any and all items manufactured and painted by automobile sub-suppliers, frame rails, commercial trucks and truck bodies, including but not limited to beverage bottles, utility bodies, ready mix concrete delivery vehicle bodies, waste hauling vehicle bodies, and fire and emergency vehicle bodies, as well as any potential attachments or components to such truck bodies, buses, farm and construction equipment, truck caps and covers, commercial trailers, consumer trailers, recreational vehicles, including but not limited to, motor homes, campers, conversion vans, vans, pleasure vehicles, pleasure craft snow mobiles, all terrain vehicles, personal watercraft, motorcycles, bicycles, boats, and aircraft. The substrate further includes industrial and commercial new construction and maintenance thereof; cement and wood floors; walls of commercial and residential structures, such office buildings and homes; amusement park equipment; concrete surfaces, such as parking lots and drive ways; asphalt and concrete road surface, wood substrates, marine surfaces; outdoor structures, such as bridges, towers; coil coating; railroad cars; printed circuit boards; machinery; OEM tools; signage; fiberglass structures; sporting goods; golf balls; and sporting equipment.

The novel compositions of this invention are also suitable as clear or pigmented coatings in industrial and maintenance coating applications.

These and other features and advantages of the present invention will be more readily understood, by those of ordinary skill in the art from the following examples.

The following Examples illustrate the invention. All parts and percentages are on a weight basis unless otherwise noted.

### EXAMPLES

The following graft copolymer with segmented arms was prepared and used to form lacquer coating compositions.

### Graft Copolymer Example 1

The graft copolymer was prepared in a 3-step process.

Step 1. Preparation of HEMA/EHMA Macromonomer, 50/50% by Weight

This illustrates the preparation of a macromonomer with primary hydroxyl groups that can be used to form the A segment (outer segment) of a segmented arm for a graft copolymer of this invention.

A 12-liter flask was equipped with a thermometer, stirrer, addition funnels, heating mantel, reflux condenser and a means of maintaining a nitrogen blanket over the reactants.

The flask was held under nitrogen positive pressure and the following ingredients were employed.

Weight
(gram)

Portion 1

| | |
|---|---|
| Methyl propyl ketone | 1581.7 |
| 2-Hydroxyethyl methacrylate (HEMA) | 565.53 |
| 2-Ethylhexyl methacrylate (EHMA) | 565.53 |

Portion 2

| | |
|---|---|
| Diaquabis(borondifluorodiphenyl glyoximato) cobaltate (II), | 1.414 |
| Co(DPG-BF₂) | |
| Acetone | 177.5 |

Portion 3

| | |
|---|---|
| 2,2′-Azobis(methylbutyronitrile) (Vazo ® 67 by DuPont Co., | 9.77 |
| Wilmington, DE) | |
| Methyl propyl ketone | 107 |

Portion 4

| | |
|---|---|
| 2-Hydroxyethyl methacrylate (HEMA) | 2262.12 |
| 2-Ethylhexyl methacrylate (EHMA) | 2262.12 |

Portion 5

| | |
|---|---|
| 2,2′-Azobis(methylbutyronitrile) (Vazo ® 67 by DuPont Co., | 97.68 |
| Wilmington, DE) | |
| Methyl propyl ketone | 1070 |
| Total | 8700.36 |

Portion 1 mixture was charged to the flask and the mixture was heated to reflux temperature and refluxed for about 20 minutes. Portion 2 and Portion 3 solution were added through separate addition funnels over 10 minutes and the reaction mixture was refluxed for 10 minutes. Portion 4 was fed to the flask over 240 minutes while Portion 5 was simultaneously fed to the flask over 270 minutes, and the reaction mixture was held at reflux temperature throughout the course of additions. Reflux was continued for another 2 hours and the solution was cooled to room temperature and filled out. The resulting macromonomer solution was a light yellow clear polymer solution and had a solid content of about 63.2% and a Gardner-Holtz viscosity of C. The macromonomer had a 3,356 Mw and 2,383 Mn.

Step 2. Preparation of an AB Segmented Macromonomer BMA/MMA//HEMA/EHMA, 35/50//7.5/7.5% by Weight

This shows the preparation of a segmented macromonomer where the B segment (inner segment) has no specific functional groups but a relatively high calculated Tg of 64.6° C. and the A segment (outer segment) contains primary hydroxyl groups and a relatively low calculated Tg of 18.9° C., from the macromonomer prepared above. It was then used to form the entire segmented arm of the graft copolymer of this invention.

A 5-liter flask was equipped as above. The flask was held under nitrogen positive pressure and the following ingredients were employed.

Weight (gram)

Portion 1

| | |
|---|---|
| Macromonomer (prepared in Step 1) | 406.15 |
| Methyl propyl ketone | 681.1 |

Portion 2

| | |
|---|---|
| Methyl methacrylate (MMA) | 880.0 |
| Butyl methacrylate (BMA) | 616.0 |

Portion 3

| | |
|---|---|
| t-Butyl peroctoate (Elf Atochem North America, Inc., | 30.0 |
| Philadelphia, PA) | |
| Methyl propyl ketone | 320.0 |
| Total | 2933.25 |

Portion 1 mixture was charged to the flask and the mixture was heated to reflux temperature and refluxed for about 10 minutes. Portion 2 was added over 3 hours and Portion 3 was simultaneously added over 3.5 hours while the reaction mixture was held at reflux temperature. The reaction mixture was refluxed for another 1.5 hours. After final cooling, the resulting macromonomer solution was clear and had a solid content of about 59.4% and a Gardner-Holtz viscosity of Z. The macromonomer had a 13,886 Mw and 7,485 Mn.

Step 3. Preparation of a Graft Copolymer with Segmented Arms

This shows the preparation of a graft copolymer of this invention containing primary hydroxyl groups and carboxylic acid groups on the backbone, primary hydroxyl groups on the A segment (outer segment) of the arm, and no specific functional groups on the B segment (inner segment) of the arm, specifically methyl methacrylate-co-butyl acrylate-co-hydroxyethyl acrylate-co-acrylic acid-g-butyl methacrylate-co-methyl methacrylate-b-hydroxyethyl methacrylate-co-ethyl hexyl methacrylate, 30/20/6/4//14/20/3/3% by weight, from a macromonomer prepared above. The A segment is relatively short, and the B segment has a relatively high calculated Tg.

A 2-liter flask was equipped as above. The flask was held under nitrogen positive pressure and the following ingredients were employed.

Weight (gram)

Portion 1

| | |
|---|---|
| Segmented Macromonomer (prepared in Step 2) | 466.7 |
| Ethyl acetate | 185.9 |

Portion 2

| | |
|---|---|
| Methyl methacrylate | 210.0 |
| Butyl acrylate | 140.0 |
| Hydroxyethyl acrylate | 42.0 |
| Acrylic acid | 28.0 |

Portion 3

| | |
|---|---|
| t-Butyl peroctoate (Elf Atochem North America, Inc., | 8.75 |
| Philadelphia, PA) | |
| Ethyl acetate | 158 |

Portion 4

| | |
|---|---|
| t-Butyl peroctoate (Elf Atochem North America, Inc., | 1.75 |
| Philadelphia, PA) | |
| Ethyl acetate | 31.6 |
| Total | 1272.7 |

Portion 1 mixture was charged to the flask and the mixture was heated to reflux temperature and refluxed for about 10 minutes. Portion 2 and 3 were simultaneously added over 3 hours while the reaction mixture was held at reflux temperature. The reaction mixture was refluxed for 30 minutes. Portion 4 was added over 5 minutes, and the reaction mixture was refluxed for another 2 hours. After final cooling, the resulting graft copolymer solution was clear and had a solid content of about 56.2% and a Gardner-Holtz viscosity of Z2¼. The graft copolymer had a 51,356 Mw and 14,001 Mn, and a Tg of 45.3° C. measured by Differential Scanning Calorimetry.

### Basecoat Lacquer Comparative Example 1 and Examples 2-3

### Basecoat Preparation

Three red metallic basecoat lacquers (Basecoat Lacquers of Comparative Example 1 and Examples 2 and 3) were prepared by mixing together following components listed in Table 1 in an air mixer in the order shown.

TABLE 1

| | Weight (grams) | | |
|---|---|---|---|
| | Comp. | | |
| Component | Ex. 1 | Ex. 2 | Ex. 3 |
| Red Metallic Composite Tinting Solution¹ | 518.42 | 518.42 | 518.42 |
| ChromaPremier ® 62320F Basecoat Binder² | 453.3 | | |
| ChromaSystems ® 7175S Basemaker³ | 828.27 | | |
| Graft Copolymer with Segmented Arms⁴ | | 69.42 | 45.13 |
| Highly Branched Copolyester Polyol⁵ | | | 21.01 |
| Organic Solvent Blend⁶ | | 956 | 960 |

Table Footnotes
¹The Red Metallic Composite Tinting Solution was produced by mixing together, on an air mixer, 7884.55 grams of DuPont MasterTint ® Magenta Tinting (864J) with 1010.06 grams of DuPont MasterTint ® Medium Coarse (813J), all supplied by DuPont Company, Wilmington, Delaware.
²ChromaPremier ® 62320F Basecoat Binder is supplied by DuPont Company, Wilmington, Delaware.
³ChromaSystems ® 7175S Basemaker is supplied by DuPont Company, Wilmington, Delaware.
⁴Graft copolymer Example 1.
⁵The highly branched polyester polyol was prepared in accordance with the procedure described in Resin Solution 5 of WO 03/070843 published Aug. 28, 2003 at page 23, line 21 to page 24, line 19 but made in methyl amyl ketone as the solvent versus propylene glycol monomethyl ether acetate.
⁶The Solvent blend was prepared by mixing together, on an air mixer, 7964.60 grams of butyl acetate with 3413.40 grams of methyl amyl ketone.

The resulting basecoats were then applied individually to cold roll steel panels by following procedure.

### Panel Preparation and Testing

DuPont Variprime® Self-Etching Primer was prepared by mixing together 600 grams of 615S Variprime® with 400 grams of 616S Converter, all supplied by DuPont Company, Wilmington, Del. The Self-Etching Primer was sprayed according to the instructions in the ChromaSystem™ Technical Manual supplied by DuPont Company, Wilmington, Del. over cold rolled steel panels (sanded with Norton 80-D sandpaper supplied by Norton, Worcester, Mass., and wiped twice with DuPont 3900S First Klean™ supplied by DuPont Company, Wilmington, Del.) resulting in a film thickness of 25.4 to 28 micrometers (1.0 to 1.1 mils). The basecoats (Examples 1-2 and Comparative Example 3) were then applied per the ChromaPremier® Basecoat instructions in the ChromaSystem™ Technical Manual, resulting in film thicknesses of 28 to 30 micrometers (1.1 to 1.2 mils). After flashing, 72200S ChromaPremier® Productive Clear (528 grams 72200S ChromaPremier® Productive Clear blended with 187 grams 12305S ChromaPremier® Activator and 185 grams 12375S ChromaPremier® Medium Reducer, all supplied by DuPont Company, Wilmington, Del.) was applied per the instructions in the ChromaSystem™ Technical Manual, resulting in a film thickness of about 56 micrometers (2.2 mils). After flashing, the panels were baked for 20 minutes at 60° C. (140° F.). The panels were then aged for one week at approximately 25° C. @ 50% relative humidity prior to testing.

The coating compositions were then tested for chipping, humidity resistance, adhesion, and appearance. The following test procedures were used for generating the data reported in the Table below.

Chip resistance was measured with a gravelometer under the procedure described in ASTM-D-3170-87 using a 55° panel angle with panels and stones kept in the freezer for a minimum of 2 hours prior to chipping (panels were tested with 0.47 liter (1 pint)/1.42 liters (3 pints) of stones after a 30 minute at 60° C. (140° F.) bake then air drying for an additional 7 days (dry chip test) and also baking for 30 minutes at 60° C. (140° F.) then air drying for an additional 7 days followed by an additional 96 hours in a humidity cabinet (ASTM-D-2247-99) at 100% relative humidity (wet chip test).

Gloss was measured at 20° and 60° using a Byk-Gardener Glossmeter.

Distinctness of Image (DOI) was measured using a Dorigon II (HunterLab, Reston, Va.).

Cross (X) hatch and grid hatch adhesion to underlying and overlying coating layers was determined using test method ASTM D3359 after initial cure and then after 96 hours in the humidity cabinet (ASTM-D-2247-99) at 100% relative humidity.

### Test Results

Below in Table 2 are the gloss (using a BYK-Gardner glossmeter) and distinctness of image (using a Dorigon II meter) values:

TABLE 2

| | 20° Gloss | DOI |
|---|---|---|
| Basecoat | BC/CC | BC/CC |
| Comp. Ex. 1 | 86.8 | 89 |
| Ex. 2 | 84.2 | 90.9 |
| Ex. 3 | 86.4 | 85.1 |

This data shows that the use of branched polymers with segmented arms in the lacquer basecoat did not adversely affect appearance.

The basecoat/clear coat panels were subjected to the chip resistance test described earlier. The results are shown in Table 3 below:

TABLE 3

Chip Resistance

| Basecoat* | 1 Pint | 3 Pints |
|---|---|---|
| Comp. Ex. 1 | 5 | 4.5 |
| Ex. 2 | 6 | 5 |
| Ex. 3 | 7 | 6 |

*All basecoats were further coated with the clear coat described above in panel preparation.

The data showed that the panels' chip performance significantly benefited from the use of branched polymers with segmented arms in the lacquer basecoat.

Table 4 below shows the results of the X-hatch and grid hatch adhesion test (ASTM D3359), DOI readings after 96 hours in the humidity cabinet (ASTM-D-2247-99) at 100% relative humidity. Readings were taken before exposure (initially), and immediately after removal from the humidity cabinet (wet).

TABLE 4

| | X-Hatch Adhesion | | Grid Hatch Adhesion | | DOI |
|---|---|---|---|---|---|
| Basecoat* | Initial | Wet | Initial | Wet | Wet |
| Comp. Ex. | 9.5 | 9 | 10 | 9.5 | 49.3 |
| Ex. 2 | 9.5 | 9.5 | 9.5 | 9.5 | 67.1 |
| Ex. 3 | 10 | 9.5 | 10 | 9.5 | 75.5 |

*All basecoats were further coated with the clear coat described above in panel preparation.

The data showed that the panels' moisture resistance benefited from the use of the branched polymers with segmented arms in the lacquer basecoat and the adhesion to other coating layers was not impaired.

Another graft copolymer with segmented arms was prepared and used to form another lacquer coating composition.

### Graft Copolymer Example 2

As in the GRAFT COPOLYMER EXAMPLE 1 it was prepared in a 3-step process.

Step 1. Preparation of HEMA/BMA Macromonomer, 50/50% by Weight

This illustrates the preparation of a macromonomer with primary hydroxyl groups that can be used to form the A segment (outer segment) of a segmented arm for a graft copolymer of this invention.

A 12-liter flask was equipped as in GRAFT COPOLYMER EXAMPLE 1. The flask was held under nitrogen positive pressure and the following ingredients were employed.

Weight
(gram)

Portion 1

| | |
|---|---|
| Methyl propyl ketone | 1581.7 |
| 2-Hydroxyethyl methacrylate (HEMA) | 565.53 |
| Butyl methacrylate (BMA) | 565.53 |

Portion 2

| | |
|---|---|
| Diaquabis(borondifluorodiphenyl glyoximato) cobaltate (II), | 1.414 |
| Co(DPG-BF₂) | |
| Acetone | 177.5 |

Portion 3

| | |
|---|---|
| 2,2′-Azobis(methylbutyronitrile) (Vazo ® 67 by DuPont Co., | 9.77 |
| Wilmington, DE) | |
| Methyl propyl ketone | 107 |

Portion 4

| | |
|---|---|
| 2-Hydroxyethyl methacrylate (HEMA) | 2262.12 |
| Butyl methacrylate (BMA) | 2262.12 |

Portion 5

| | |
|---|---|
| 2,2′-Azobis(methylbutyronitrile) (Vazo ® 67 by DuPont Co., | 97.68 |
| Wilmington, DE) | |
| Methyl propyl ketone | 1070 |
| Total | 8700.36 |

The procedure of Step 1 of GRAFT COPOLYMER EXAMPLE 1 was repeated. The resulting macromonomer solution was a light yellow clear polymer solution and had a solid content of about 62.4% and a Gardner-Holtz viscosity of d. The macromonomer had a 3,009 Mw and 2,181 Mn.

Step 2. Preparation of an AB Segmented Macromonomer BMA/MMA//HEMA/BMA, 30/50//10/10% by Weight

This shows the preparation of a segmented macromonomer where the B segment (inner segment) has no specific functional groups but a relatively high calculated Tg of 67.9° C. and the A segment (outer segment) contains primary hydroxyl groups and a relatively low calculated Tg of 36.5° C., from the macromonomer prepared above. It was then used to form the entire segmented arm of the graft copolymer of this invention.

A 5-liter flask was equipped as in Step 2 of GRAFT COPOLYMER EXAMPLE 1. The flask was held under nitrogen positive pressure and the following ingredients were employed.

Weight
(gram)

Portion 1

| | |
|---|---|
| Macromonomer (prepared in Step 1 of Graft Copolymer Ex. 1) | 788.10 |
| Methyl propyl ketone | 571.34 |

Portion 2

| | |
|---|---|
| Methyl methacrylate (MMA) | 1232.0 |
| Butyl methacrylate (BMA) | 739.0 |

Portion 3

| | |
|---|---|
| t-Butyl peroctoate (Elf Atochem North America, Inc., | 42.0 |
| Philadelphia, PA) | |
| Methyl propyl ketone | 448.0 |
| Total | 3820.44 |

The procedure of Step 2 of GRAFT COPOLYMER EXAMPLE 1 was repeated. After final cooling, the resulting macromonomer solution was clear and had a solid content of about 64.5% and a Gardner-Holtz viscosity of Z4½. The macromonomer had a 10,437 Mw and 6,216 Mn.

Step 3. Preparation of a Graft Copolymer with Segmented Arms

This shows the preparation of a graft copolymer of this invention containing primary hydroxyl groups and carboxylic acid groups on the backbone, primary hydroxyl groups on the A segment (outer segment) of the arm, and no specific functional groups on the B segment (inner segment) of the arm, specifically methyl methacrylate-co-butyl acrylate-co-hydroxyethyl acrylate-co-acrylic acid-g-butyl methacrylate-co-methyl methacrylate-b-hydroxyethyl methacrylate-co-butyl methacrylate, 30/20/6/4//12/20//4/4% by weight, from a macromonomer prepared above. The A segment is relatively short, and the B segment has a relatively high calculated Tg.

A 2-liter flask was equipped as in Step 3 of GRAFT COPOLYMER EXAMPLE 1. The flask was held under nitrogen positive pressure and the following ingredients were employed.

Weight (gram)

Portion 1

| | |
|---|---|
| Segmented Macromonomer (prepared in Step 2 above) | 430.8 |
| Ethyl acetate | 221.8 |

Portion 2

| | |
|---|---|
| Methyl methacrylate | 210.0 |
| Butyl acrylate | 140.0 |
| Hydroxyethyl acrylate | 42.0 |
| Acrylic acid | 28.0 |

Portion 3

| | |
|---|---|
| t-Butyl peroctoate (Elf Atochem North America, Inc., | 8.75 |
| Philadelphia, PA) | |
| Ethyl acetate | 158 |

Portion 4

| | |
|---|---|
| t-Butyl peroctoate (Elf Atochem North America, Inc., | 1.75 |
| Philadelphia, PA) | |
| Ethyl acetate | 31.6 |
| Total | 1272.7 |

The procedure of the Step 3 of GRAFT COPOLYMER EXAMPLE! was repeated. After final cooling, the resulting graft copolymer solution was clear and had a solid content of about 55.2% and a Gardner-Holtz viscosity of Z1. The graft copolymer had a 45,724 Mw and 14,172 Mn, and a Tg of 47.1° C. measured by Differential Scanning Calorimetry.

### Basecoat Lacquer Comparative Example 4 and Example 5

### Basecoat Preparation

A Solvent Blends A and B were prepared by mixing the following ingredients on an air mixer:

Solvent Blend A

| Component | Grams |
|---|---|
| Acetone | 162 |
| Isobutyl alcohol | 234 |
| Isopropanol | 180 |
| Methyl amyl ketone | 612 |
| Methyl Isobutyl Ketone | 108 |
| Aliphatic hydrocarbon (bp = 90-110 C.) | 270 |
| Xylene | 216 |
| Aromatic hydrocarbon (bp = 150-190 C.) | 18 |
| Total | 1800 |

Solvent Blend B

| Component | Grams |
|---|---|
| Butyl acetate | 7964.60 |
| Methyl amyl ketone | 3413.40 |
| Total | 11378.00 |

A CAB Solution, shown below, was produced by slowly adding cellulose acetate butyrate to solvent while mixing on an air mixer:

| Component | Description | Grams |
|---|---|---|
| Solvent Blend B | Solvent Blend | 5055.57 |
| CAB-381-2* | cellulose acetate butyrate | 669.12 |
| CAB-531-1* | cellulose acetate butyrate | 223.04 |
| | Total | 5947.73 |

*Supplied by Eastman Chemical Co., Kingsport, Tennessee

Basecoat lacquer coating compositions, without pigmentation, were made to test the compatibility of the binder components. The formulations were:

| | Weight (grams) | |
|---|---|---|
| | Comp. | Ex. |
| Component | Ex 4 | 5 |
| CAB solution (from above) | 100 | 100 |
| Standard Low Molecular weight Hydroxyl functional | 15.48 | 15.48 |
| Acrylic - 66% solids in Butyl acetate | | |
| Segmented acrylic (Resin Example 2) | 67.27 | 67.27 |
| Highly Branched Copolyester Polyol-Same | 30.77 | 0 |
| composition as Solution 5 of FA-1061 but made in | | |
| Methyl Amyl Ketone as the solvent vs. propylene | | |
| glycol monomethyl ether acetate | | |
| Solvent blend A (from above) | 196.48 | 127.25 |

### Panel Preparation & Testing

Each of the lacquer coating compositions above was applied with a doctor blade over a separate glass panel to a dry coating thickness of approximately 40-50 micrometers and air dried at ambient temperature conditions.

The coating compositions were inspected for visual appearance and rated on a scale of 1=worst to 10=best for appearance. They were also tested for haze by measuring the light transmitted through the film using a Hunter Lab—Color Quest measuring device. Haze was calculated by the following equation:
Haze=(diffuse transmittance×100)/total transmittance

These coatings were tested initially and after aging for 1 month at 43 C. The results were:

| | Ex #1 - | Ex#1 - | Ex#2 - | Ex#2 - |
|---|---|---|---|---|
| | Initial | Aged | Initial | Aged |
| Haze | 1.75 | 0.62 | 1.98 | 1.26 |
| Visual rating | 10 | 10 | 10 | 10 |

The compositions that include acrylic alone and acrylic/polyester blends have practically the same values for haze and appearance. These similar and low haze values and excellent visual ratings show the unexpectedly excellent compatibility of the high molecular weight segmented acrylic with a high molecular weight polyester, both initially and after aging. This is a significant advantage of these types of segmented acrylics.

Various modifications, alterations, additions or substitutions of the compositions and process of this invention will be apparent to those skilled in the art without departing from the spirit and scope of this invention. This invention is not limited by the illustrative embodiments set forth herein, but rather is defined by the following claims.

What is claimed is:

1. A lacquer coating composition, comprising a graft copolymer with segmented side arm(s), wherein the graft copolymer has a polymeric backbone and segmented arm(s) formed of at least two polymeric segments, grafted onto the backbone, wherein
(a) the backbone is of polymerized ethylenically unsaturated monomers; and
(b) the segmented arm(s) are of polymerized ethylenically unsaturated monomers that are attached to the backbone via a single point,
and wherein the segments on the arm(s) are formed of substantially differing composition from those of adjacent segment(s) and differ by the presence of, type of, and/or relative concentrations of functional groups, wherein the functional groups are selected from at least one of the group consisting of acid, hydroxyl or amine groups or mixtures of hydroxyl and either acid or amine groups.

2. The lacquer of claim 1, wherein the graft copolymer has a weight average molecular weight ranging from about 5,000-100,000.

3. The lacquer of claim 1, wherein the segmented arm(s) are formed from a macromonomer having a segmented structure that is polymerized into the backbone via a single terminal ethylenically unsaturated group.

4. The lacquer of claim 1, wherein the backbone contains at least one of said functional groups but has at least one functional group which is different from the functional group(s) on the arm(s).

5. The lacquer of claim 1 wherein the segmented arm(s) are formed with two segments of either AB block or tapering architecture.

6. The lacquer of claim 5 wherein the segmented arms contain an inner segment attached directly to the backbone and an outer segment attached to the inner segment, wherein the inner segment is essentially free of functional groups and the outer segment contains at least one type of functional group concentrated thereon selected from at least one of the group consisting of acid, hydroxyl or amine groups or mixtures of hydroxyl and either acid or amine groups.

7. The lacquer of claim 6, wherein the backbone contains at least one of said functional groups but has at least one functional group which is different from the functional group(s) on the arm(s).

8. The lacquer of claim 7 wherein the backbone and arm contain hydroxyl groups and the backbone additionally contains a carboxylic acid group.

9. The lacquer of claim 6, wherein the inner arm segment is hard relative to the outer arm segment and has a calculated Tg of at least 30° C.

10. The lacquer of claim 9, wherein the outer arm segment is soft relative to the inner arm segment and has a calculated Tg of no greater than 25° C.

11. The lacquer of claim 1, wherein the graft copolymer is prepared from polymerized acrylic monomers and optionally styrene.

12. A lacquer coating composition comprising about 5-95% by weight of a film-forming binder and correspondingly about 95-5% by weight of a volatile organic liquid carrier, wherein the binder contains a graft copolymer with segmented arm(s), wherein the graft copolymer has a weight average molecular weight ranging from about 5,000-100,000, a polymeric backbone and segmented arm(s) comprising on average two polymeric segments, grafted onto the backbone, wherein
(a) the backbone is of polymerized ethylenically unsaturated monomers; and
(b) the segmented arm(s) are macromonomers formed of polymerized ethylenically unsaturated monomers that are attached to the backbone via a single terminal ethylenically unsaturated group,
wherein the segments on the arm(s) are formed of substantially differing composition and differ by the presence of, type of, and/or relative concentrations of functional groups, wherein the functional groups are selected from at least one of the group consisting of acid, hydroxyl or amine groups or mixtures of hydroxyl and either acid or amine groups,
and the backbone contains at least one of said functional groups but has at least one functional group which is different from the functional group(s) on the arm(s).

13. The lacquer of claim 1 wherein the graft copolymer comprises 30% to 70% by weight, based on the weight of the polymer, of segmented arms of polymerized ethylenically unsaturated monomers and 70% to 30% by weight, based on the weight of the polymer of a backbone of polymerized ethylenically unsaturated monomers.

14. The lacquer of claim 1 wherein the functional ethylenically unsaturated monomers used to form the backbone and arm(s) are selected from the group consisting of hydroxy alkyl (meth)acrylates having 1-4 carbon atoms in the alkyl group, ethylenically unsaturated carboxylic acids, and any mixtures thereof, and the backbone and the macromonomers contain additional monomers selected from the group consisting of alkyl (meth)acrylates having 1-20 carbon atoms in the alkyl group, cycloaliphatic (meth)acrylates, styrene, alpha methyl styrene, vinyl toluene, acrylonitrile, methacrylonitrile, acrylamide, methacrylamide, isobornyl (meth)acrylate and any mixtures thereof.

15. The lacquer of claim 6 wherein the macromonomers consist essentially of polymerized monomers of ethyl hexyl methacrylate, hydroxy ethyl (meth)acrylate and the monomers of the backbone consist essentially of methyl methacrylate, hydroxy ethyl acrylate, acrylic acid, and butyl acrylate.

16. The lacquer of claim 1 or 12 wherein said lacquer comprises an acrylic polymer, polyester, a highly branched copolyester polyol, alkyd resin, acrylic alkyd resin, cellulose acetate butyrate, an iminated acrylic polymer, ethylene-vinyl acetate co-polymer, nitrocellulose, plasticizer or a combination thereof.

17. The lacquer of claim 1 or 12 wherein said lacquer further comprises metallic driers, chelating agents, or a combination thereof.

18. The lacquer of claim 1 or 12 comprising a pigment, flake or a combination thereof.

19. The lacquer of claim 1 or 12, wherein said lacquer further comprises as part of the binder, a crosslinking agent.

20. A process for producing a coating on the surface of a substrate, said process comprising:
applying a layer of a lacquer of claim 1 or 11 on said surface; and
drying said layer to form said coating on said surface of said substrate.

21. The process of claim 20 further comprising applying a layer of clear coating composition over said layer of said lacquer.

22. The process of claim 21 wherein said lacquer is a pigmented basecoat composition.

23. The process of claim 20 wherein said drying step takes place under ambient conditions.

24. The process of claim 20 wherein said drying step takes place at elevated temperatures.

25. The process of claim 20 wherein said lacquer is a pigmented basecoat or a clearcoat composition.

26. A coated substrate produced in accordance with the process of claim 20.
